(19) United States
(12) Patent Application Publication
(10) Pub. No.: US 2006/0170918 A1
(43) Pub. Date: Aug. 3, 2006

(54) Detection Apparatus and Detection Method for Plasmon Resonance and Fluorescence

(75) Inventor: Satoru Nishiuma, Kawasaki-shi (JP)

Correspondence Address: FITZPATRICK CELLA HARPER & SCINTO, 30 ROCKEFELLER PLAZA, NEW YORK, NY, 10112, US

(73) Assignee: Canon Kabushiki Kaisha, Tokyo (JP)

(21) Appl. No.: 11/275,746

(22) Filed: Jan. 26, 2006

(30) Foreign Application Priority Data
Jan. 31, 2005 (JP) 023517/2005(PAT.)

(51) Int. Cl. G01J 3/30; G01N 21/55; G01N 33/543

(52) U.S. Cl. 356/318; 356/445; 436/518

(57) ABSTRACT

A plurality of different ligands are fixed on a metal array including a plurality of metal films formed on a substrate and are irradiated with light from a rear surface of the substrate to measure surface plasmon resonance and fluorescence based on evanescent wave at the same time, thus permitting screening for concurrently measuring a plurality of items with accuracy.

### FIELD OF THE INVENTION AND RELATED ART

The present invention relates to a detection apparatus and a detection method for quickly finding molecules leading to pathogenic bacteria to perform diagnosis by screening a plurality of biomolecules such as protein and DNA at the same time.

In blood, there are a plurality of makers for specific diseases such as cancer, hepatitis, diabetes, and osteoporosis. When a person contracts a disease, a concentration of a specific protein is increased compared with that in ordinary times. It is possible to early detect means disease by monitoring the markers even in ordinary times, so that the monitoring of the markers is expected as a next-generation medical technology.

One of methods for analyzing raw and crude protein is based on a sensor for identifying a specific compound by utilizing a biological ligand-target substance interaction. A combination of ligand-target substance may include an antigen-antibody composite utilizing a connection between proteins, a DNA composite comprising a connection between nucleic acid and its complementary substance, and a composite of receptors.

As the sensor, there are those of some types such as fluorescence immunoassay method, plasmon resonance method, and light interference method. In either case, such a common step that a ligand is fixed on a sensor surface and a target substance in a specimen is selectively screened with high sensitivity and connected with the ligand to remove impurities, so that only an objective protein is effectively fixed on a surface of substrate.

In the fluorescence immunoassay method, a second ligand labeled with a fluorescent colorant is further connected with the ligand-target substance composite to excite the fluorescent colorant, and an amount of fluorescence is measured to determine a concentration of the target substance. In the plasma resonance method, a concentration of a target substance connected with a ligand fixed on a surface of a metal film or metal fine particles is measured by utilizing such a property that metal plasmon is responsive to a change in refractive index of an interfacial substance with high sensitivity.

However, the fluorescence immunoassay method can measure the concentration of the target substance with high sensitivity but fails to measure a reaction speed. On the other hand, the plasmon resonance method can measure the response speed but it is somewhat difficult to measure the response speed with high sensitivity or a concentration of a low-molecular weight compound.

As a method for overcoming these problems, a method in which a fluorescence immunoassay method and a surface plasmon resonance method are performed concurrently to remedy the problems of both the fluorescence immunoassay method and the surface plasmon resonance has been proposed in U.S. Pat. No. 6,194,223 and Japanese Laid-Open Patent Application (JP-A) No. 2002-62255.

However, in the method in which the fluorescence immunoassay method and the surface plasmon resonance are performed concurrently disclosed in U.S. Pat. No. 6,194,223 and JP-A 2002-62255, a ligand to be fixed is one species. Accordingly, only one species of target substance can be measured by one measurement. On the other hand, oncogenic markers which have been currently found and less specific to sites or regions. For example, the oncogenic markers such as CEA and CA19-9 are formed of cells of a plurality of organs such as stomach, large bowel, pancreas, and lung, so that there has arisen such a problem that it is impossible to specify which site is cancerous only by measuring one species of marker.

### SUMMARY OF THE INVENTION

An object of the present invention is to provide a detecting apparatus having solved the above described problem.

Another object of the present invention to provide a detection method having solved the above described problem.

According to an aspect of the present invention, there is provided a detection apparatus, comprising:

a substrate;

a metal array, formed on the substrate, comprising a plurality of metal films;

a first ligand, disposed on the metal films, for catching a first chemical substance;

a second ligand, disposed on the metal films, for catching a second chemical substance different from the first chemical substance;

a first optical system disposed on a first surface of the substrate; and

a second optical system disposed on a second surface, of said substrate, different from the first surface;

wherein the first optical system includes first detection means comprising a two-dimensional optical sensor for detecting reflected light when a ligand-target substance composite comprising the metal films, the first and second ligands, and the first and second chemical substances is irradiated with emitted light from a first light source; and

wherein the second optical system includes second detection means comprising a two-dimensional optical sensor for detecting fluorescence from the ligand-target substance composite excited when the ligand-target substance composite is irradiated with the emitted light from the first light source.

According to another aspect of the present invention, there is provided a detection method, comprising:

a step of catching a first chemical substance by a first ligand fixed on a plurality of metal films formed on a substrate,

a step of catching a second chemical substance, different from the first chemical substance, by a second ligand fixed on the plurality of metal films,

a step of causing emitted light from a first light source to enter a ligand-target substance composite comprising the substrate, the metal films, and the first and second chemical substances,

a first detection step of detecting reflected light from the ligand-target substance composite by a two-dimensional optical sensor, and

a second detection step of detecting fluorescence from the ligand-target substance composite excited by the emitted light caused to enter the ligand-target substance composite.

These and other objects, features and advantages of the present invention will become more apparent upon a consideration of the following description of the preferred embodiments of the present invention taken in conjunction with the accompanying drawings.

### BRIEF DESCRIPTION OF THE DRAWINGS

FIGS. 1(a) and 1(b) are schematic views showing a concurrent imaging apparatus for plasmon resonance and fluorescence in an embodiment of a detection apparatus according to the present invention.

FIG. 2 is a schematic view showing a concurrent imaging apparatus for plasmon resonance and fluorescence in Embodiment 1 of the present invention.

FIG. 3 is a perspective view showing a concurrent imaging apparatus for plasmon resonance and fluorescence in Embodiment 1 of the present invention.

FIGS. 4(a) and 4(b) are a plan view and a sectional view, respectively, schematically showing a state in which a ligand is fixed on a metal array.

FIGS. 5(a) to 5(d) are a plan view, a sectional view, a schematic view, and a schematic view, respectively, showing a state in which a ligand is fixed on a 2×2 metal array.

FIG. 6 is a schematic enlarged view showing a substrate portion of the imaging apparatus of the present invention.

FIGS. 7(a) and 7(b) are schematic views showing a luminescent state on the substrate of the detection apparatus of the present invention.

FIGS. 8(a) and 8(b) are schematic views showing a structure of a detection portion of the detection apparatus of the present invention.

FIG. 9 is a graph showing results in Embodiment 1 of the present invention.

FIG. 10 is a flow chart showing a protocol or procedure of measurement in Embodiment 1 of the present invention.

### DETAILED DESCRIPTION OF THE PREFERRED EMBODIMENTS

The present invention is directed to a detection substrate a detection apparatus and a detection method which are capable of performing detection of a plurality of ligand-target substance composites (hereinafter also simply referred to as a “ligand-target substance composite”) at the same time on the basis of the fluorescence immunoassay and the surface plasmon resonance method. Further, in the present invention, it is also possible to effect imaging on the basis of information detected through the above described detection.

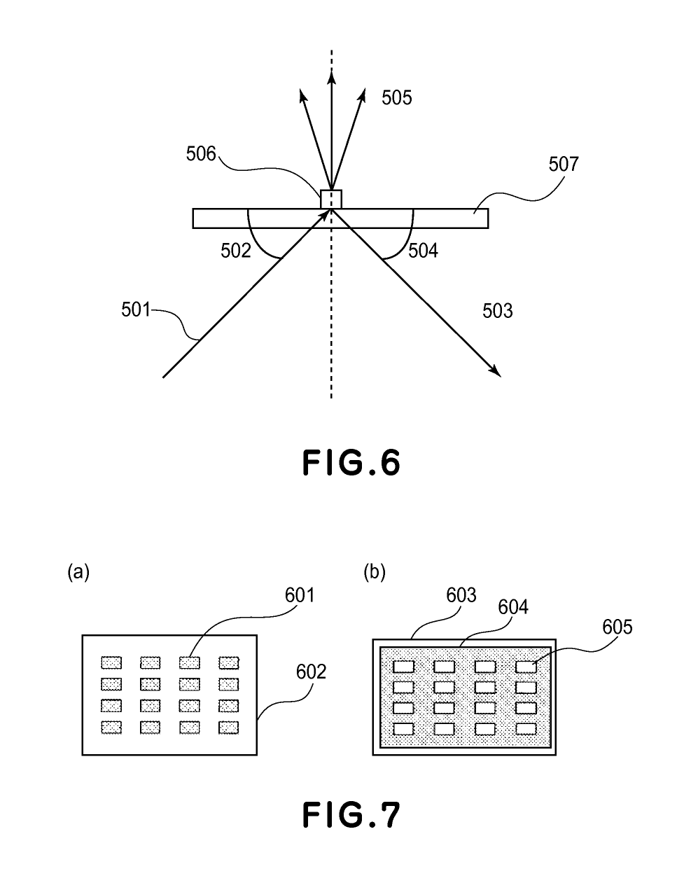

FIG. 6 is a schematic enlarged view showing a substrate portion of an imaging apparatus as the detection apparatus according to the present invention. Referring to FIG. 6, different ligands are disposed in advance on an array of metal films formed on a dielectric substrate 507, and the ligands and a specimen are caused to react with each other to form a ligand-target substance composite. Thereafter, the ligand-target substance composite is irradiated with light 501 from a back surface side of the substrate 507.

By using a two-dimensional optical sensor, reflected light 503 as surface plasmon resonance reflected from the metal film at an outgoing angle 504 is detected. The reflected light 503 as surface plasmon resonance varies in degree of a change in refractive index of the reflected light depending on a weight of molecules, i.e., species of the ligand-target substance composite fixed on island-like metal films 506. For this reason, incident angle 502 is changed, i.e., scanning is performed, so that it is equal to the outgoing angle 504 of the reflected light 503. In this manner, an angle dependency of reflected light intensity is monitored to discriminate the ligand-target substance composite.

Further, by irradiating the ligand-target substance composite with the light 501, the ligand-target substance composite fixed on the metal films 506 is excited by evanescent light through a dielectric film to produce fluorescence (fluorescent light) 505. This fluorescence is detected by a two-dimensional optical sensor.

FIG. 7(a) is a schematic view showing a luminescence state on the substrate and FIG. 7(b) is a schematic view showing a state in which the luminescence from the substrate shown in FIG. 7(a) is received by the two-dimensional optical sensor. In FIG. 7(a), fluorescence 601 from the ligand-target substance composite fixed on the island-like metal films formed on the substrate 602 is shown. In FIG. 7(b), areas (regions) 605, in which the fluorescence 601 from the substrate, of a light-receiving area 604 formed on a two-dimensional optical sensor 603 is shown.

Referring again to FIG. 6, the reflected light 503 has a constant reflection intensity from an area in which surface plasmon resonance by the ligand-target substance composite is not caused to occur, so that a received light intensity in a pixel area other than a site which receives the reflected light 503 from the metal film is substantially identical. Similarly, the fluorescence 505 is not emitted from substances other than the ligand-target substance composite, so that a received light intensity in a pixel area other than a site which receives the fluorescence 505 is substantially identical.

Two-dimensional light information detected by the two-dimensional optical sensor is displayed (imaged) on a display apparatus such as a cathode ray tube (CRT) or a liquid crystal display apparatus.

Display on the display apparatus can be performed on one picture area (screen) by combining respective pieces of the light information through an arithmetic/logic unit described later or performed on respective picture areas for respective pieces of the light information by dividing one picture area into a plurality of picture areas. As a result, it is possible to select an image to be displayed on the display apparatus depending on intended purpose.

As the two-dimensional optical sensor, it is possible to use a charge coupled device (CCD) sensor or a complementary metal oxide semiconductor (CMOS) sensor.

As the ligand, it is possible to use protein or nucleic acid. Examples of protein as the ligand may include antibody and the like, and examples of nucleic acid may include DNA, RNA, etc.

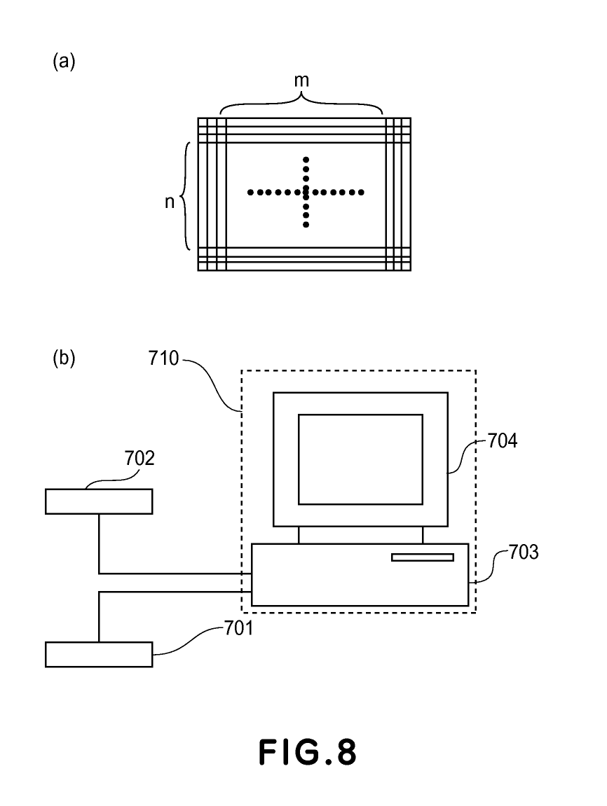

FIGS. 8(a) and 8(b) show a schematic structure of a detection portion.

FIG. 8(a) is a schematic view showing a pixel portion, constituted by n×m pixels, of a CCD area sensor. The area sensor can determine a position of pixel on the basis of electric information obtained from the area sensor, so that a signal for the pixel can be separated in the following manners 1 and 2.

1. Assuming that a positional relationship between the metal films is identical even when the substrate is replaced with another one, reflected light from a metal array consisting of island-like metal films is measured to store a pixel (information) corresponding to the metal array in advance.

2. For each detection, a position of a pixel is determined and the determined pixel (information) is stored.

Based on the information stored by either one of the above manners 1 and 2, a signal corresponding to the metal array from the reflected light due to surface plasmon resonance of the metal films or from the fluorescence due to evanescent light is separated.

In the case of the reflected light, an aspect ratio is changed, so that the aspect ratio is obtained in advance for comparison with the case of the fluorescence and then may be normalized.

FIG. 8(b) is a conceptual view of a signal separation portion for separating a signal for pixel unit from the signals from the area sensor. In the present invention, the area sensor is described by using the CCD sensor but may also be a sensor comprising light-receiving elements disposed in an array shape or the CMOS sensor.

A computer 710 for image processing is constituted by an arithmetic apparatus 703 having an arithmetic portion for determining a pixel corresponding to the metal array from signals of CCD sensors 701 and 702 and a storing portion for storing positional information of the determined pixel and (constituted) by a display apparatus 704 for displaying, on a screen, results of arithmetic computation of light information from the CCD sensors 701 and 702 performed by the arithmetic portion of the arithmetic apparatus on the basis of stored pixel information corresponding to the metal array.

In the case of displaying the computed light information from the CCD sensors 701 and 702 on the display apparatus 701, display can be performed in several manners such that respective pieces of light information are displayed on divided picture areas, that the light information from the CCD sensors 701 and 702 is subjected to the arithmetic computation at the arithmetic portion by a processing method stored in the storing portion of the arithmetic apparatus 701 in advance and is displayed as one image, and that a necessary image is displayed in every detection step. The arithmetic portion is not shown in FIG. 8(b).

Further, in order to measure surface plasmon resonance, it is possible to make the incident angle 502, between an optical axis of the light 501 emitted from the above described light source, equal to the reflection angle 504 between an optical axis of a light-receiving portion for receiving the reflected light 503. For example, this can be realized by controlling an unshown mechanism such as an arm. In the case where the arm is moved through the arithmetic portion, it is not necessary to externally input angle information during the computation of angle dependency of surface plasmon resonance into the arithmetic apparatus 703. For this reason, the arm control may preferably be performed by the arithmetic portion.

For example, the detection is principally performed in accordance with the following protocol or procedure after the substrate is set on a prism although details thereof are described later.

(1) A labeled antibody is caused to react with antigen in advance to prepare a labeled antigen-antibody pair.

(2) A fluorescence-labeled antibody is introduced into a flow channel (path) and subjected to incubation for 5 minutes.

(3) The labeled antibody is taken out and washed with phosphate buffer.

(4) A specimen in which a labeled antigen is mixed is introduced into the flow channel.

(5) Through laser light irradiation, a surface plasmon resonance curve and a charge with time in fluorescent signal are detected from respective metal film dots.

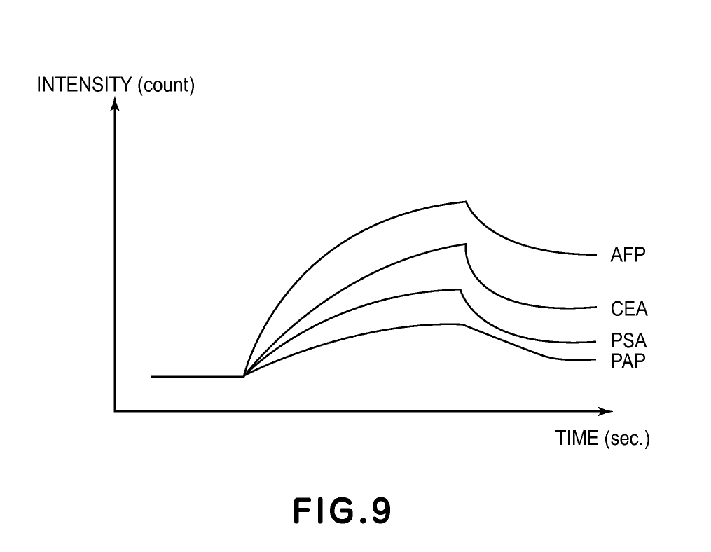

(6) When data of the change with time in fluorescent signal are plotted, a result as shown in FIG. 9 is obtained.

It is possible to perform detection for each step, as desired, to display a result thereof by controlling, through the arithmetic apparatus 703, steps including the above described steps of influent of the antibody, incubation, washing, influent of the antigen, incubation, and washing or by inputting control signals for the respective steps into the arithmetic apparatus 703.

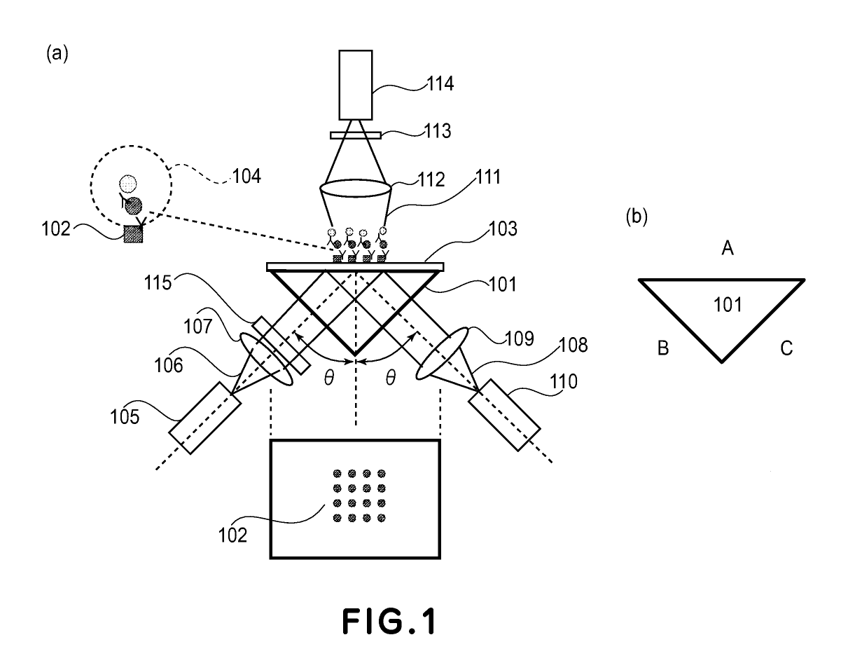

Hereinbelow, a measurement apparatus used in this embodiment according to the present invention will be described with reference to FIGS. 1(a) and 1(b). However, the present invention is not limited to the embodiment shown in FIGS. 1(a) and 1(b).

Referring to FIG. 1(b), a right-angled triangular prism 101 has three planes A, B and C. FIG. 1(a) is a schematic view showing a side surface of a measurement device and a plan view of a substrate. The measurement device is constituted by at least the prism 101, a minute metal array 102, a substrate 103, a ligand-target substance composite 104, a light source 105, laser light 106, a collimating lens 107, a polarizing filter 115, reflected light 108, a condenser lens 109, a detector 101, fluorescence 111, a condenser lens 112, a filter 113, and a detector 114.

The substrate 103 is fixed on the plane A. When the substrate 103 is viewed from above, the metal array 102 comprising a plurality of metal films (dots) on which different ligands are fixed is dispose don the substrate 103. The metal array 102 is irradiated with the laser light 106 emitted from the (laser) light source 105. The laser light 106 is converted into parallel light by the collimating lens 107 to enter the plane B of the prism 101. The laser light 106 having entered the plane B is reflected by the plane A and the reflected light 108 by surface plasmon resonance based on a specimen fixed on the ligands on the substrate 103 goes out of the plane C of the prism 101.

The outgoing reflected light 108 through the plane C of the prism 101 is condensed by the condenser lens 109 and enters the detector 110 comprising a two-dimensional optical sensor.

On the minute metal array 102 formed on the substrate 103, different ligands are fixed. The specimen caught by the different ligands fixed on the minute metal array 102 is excited by the laser light 106 having entered the plane A of the prism 101 to produce the fluorescence 111. The fluorescence 111 is condensed by the condenser lens 112 and only a component thereof having a predetermined wavelength enters the detector 110.

In this embodiment shown in FIGS. 1(a) and 1(b), the metal array 102 is formed on the substrate 103 and the substrate 103 is disposed on the plane A of the prism 101. However, it is also possible to directly form the metal array 102 on the plane A of the prism 101.

The prism 101 may have a refractive index of 1.4-2.0, preferably 1.5-2.0. The minute metal array 102 may have a diameter of approximately 50 μm to 5 mm, preferably approximately 100μ, for each dot (metal film), and may have a thickness of approximately 20-300 nm, preferably approximately 50 nm. The substrate 103 may preferably have the substantially same refractive index as the prism 101. As substances constituting the ligand-target substance composite 104, it is possible to selectively use a combination of antigen and antibody and a combination of DNA and its complementary DNA. As the light source 105, it is possible to selectively use laser diode or gas laser having a wavelength of 200-1000 nm, preferably 200-800 nm.

As the collimating lenses 107 and 109, it is possible to use aspheric lens, selfoc lens, planoconvex lenses or biconvex lenses, objective lens for microscope, etc. In this embodiment, the planoconvex lens or the aspheric lens may preferably be used. As the two-dimensional optical sensor 110, it is possible to use optical sensors, comprising arranged two-dimensional pixels such as a photodiode array, a CCD array, and a CMOS array. In this embodiment, depending on a concentration of the specimen, the photodiode or the CCD array may appropriately be selected.

As the condenser lenses 109 and 112, it is possible to use planoconvex cylinder lens, planoconvex cylinder lens, aspheric lens, planoconvex lenses or biconvex lenses, objective lens for microscope, and selfoc lens. In this embodiment, the planoconvex cylinder lens or the planoconcave lens may preferably be used.

As the optical filter 113, it is possible to use a color glass filter, a dichroic filter, a gelatin filter, a long-path filter, a visible light-blocking filter, and IR-pass filter. In this embodiment, the dichroic filter or the color glass filter may preferably be used. As the filter 115, a polarizing filter is used.

Hereinbelow, the present invention will be described more specifically based on Embodiments but is not limited thereto.

### Embodiment 1

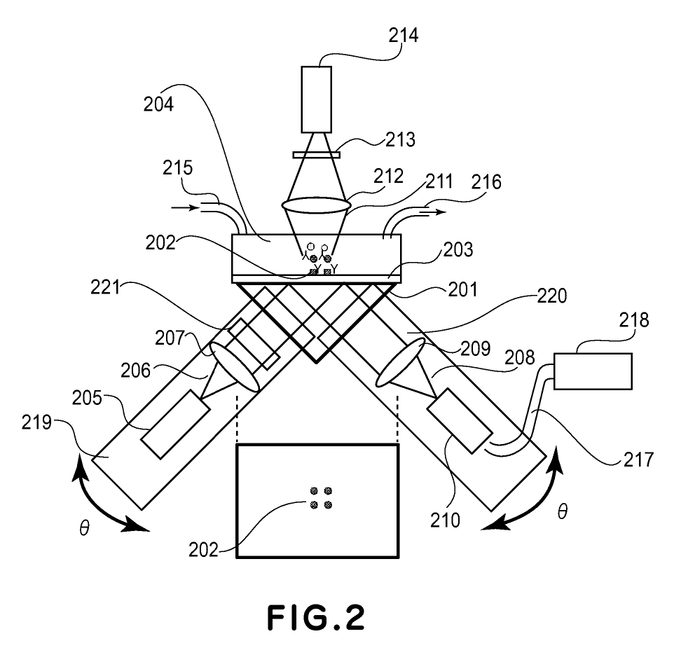

Referring to FIG. 2, onto a plane A of a prism 201 (“BK7”; A=28.3 mm, B=C=20 mm), an index matching fluid (“F-IMF-105”, mfd. by Newport Corp.) having the same refractive index as the prism 201 was applied and a substrate 203 on which a metal array 202 comprising a plurality of dots (metal films) each having a diameter of 100 μm was formed was caused to hermetically contact the prism 201.

To the substrate 203, a transparent reaction cell 204 in which a specimen and a washing liquid were introduced was bonded. As a light source 205, a laser diode (“DL3038-033”, mfd. by SANYO Electric Co., Ltd.) was used. As a collimating lens 207, a planoconvex lens (mfd. by SIGMA KOKI Co., Ltd.; diameter 5 mm) was used. As a filter 221, a polarizing filter (“SPF-30C-32”, mfd. by SIGMA KOKI Co., Ltd.) was used.

This incident optical system was fixed on an arm 219. As an optical sensor 210, a CCD area image sensor (“S7030-0906”, mfd. by Hamamatsu Photonics K.K.) was used. As a collimating lens 209, a planoconvex lens (mfd. by SIGMA KOKI Co., Ltd.) was used. This light-receiving system was fixed on an arm 220 and during measurement, scanning is effected so that an incident angle on the substrate is equal to a reflection angle from the substrate. Above the reaction cell 204, a condenser lens 213 for condensing fluorescence light (planoconvex lens mfd. by SIGMA KOKI Co., Ltd.; diameter 5 mm) and a CCD area image sensor (“S7030-0906”, mfd. by Hamamatsu Photonics K.K.) were disposed.

In order to confirm optical characteristics of the sensor, the following study was conducted.

IN a solution of Cy 5 (fluorescent dye mfd. by Amersham Biosciences, Inc.) having a concentration of 1×10⁻⁷ mol/l, the metal film array substrate 203 was immersed and then was disposed on the completed optical system. Laser light (wavelength=about 638 nm; effective intensity=3 mW; modulated with a rectangle wave of 1 kHz) was emitted from the laser diode and scanning was performed by synchronizing the arms 219 and 220 to measure a plasmon resonance characteristic and a fluorescent characteristic. As a result of the measurement, it was confirmed that these characteristics were capable of being measured with good sensitivity.

Next, detection of various antigens such as CEA, AFP, PSA and PAP which had been known as markers for cancer was tried.

First of all, on each of metal arrays, streptavidin was deposited. Thereafter, biotin-modified anti-CEA antibody, biotin-modified anti-AFP antibody, biotin-modified anti-PAS antibody, and biotin-modified anti-PAP antibody were adsorbed to prepare an immunosensor.

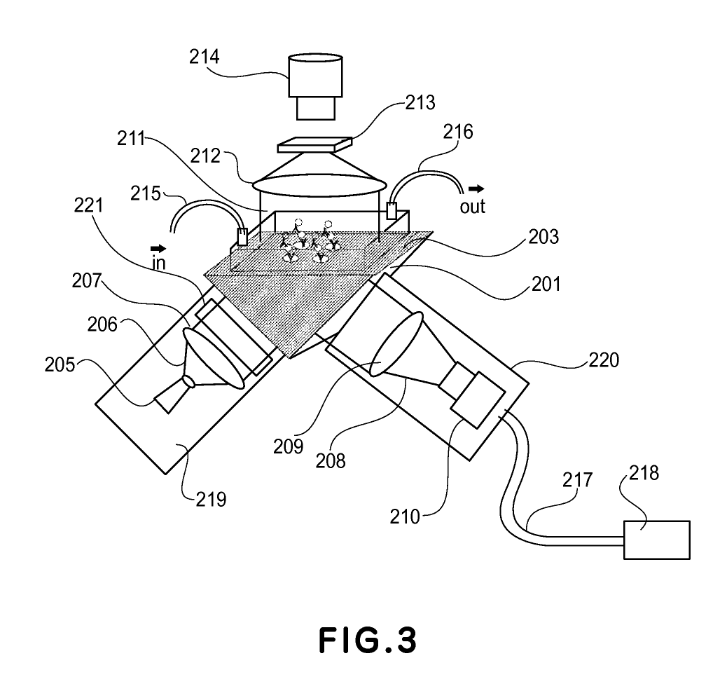

FIG. 3 is a perspective view of the optical system shown in FIG. 2, wherein the same reference numerals are used.

Similarly as in the case of FIG. 2, onto the prism 201, the index matching fluid having the same refractive index as the prism 201 was applied and the sensor 203 on which the metal array 202 of 100 μm-dia. dots was formed was caused to hermetically contact the prism 201.

To the substrate 203, the transparent reaction cell 204 in which a specimen and a washing liquid were introduced was bonded. As the light source 205, a laser diode was used. As the collimating lens 207, a planoconvex lens was used. As the filter 221, a polarizing filter was used.

This incident optical system was fixed on the arm 219. As the optical sensor 210, a CCD area image sensor was used. As the collimating lens 209, a planoconvex lens was used. This light-receiving system was fixed on the arm 220 and during measurement, scanning is effected so that an incident angle on the substrate is equal to a reflection angle from the substrate. Above the reaction cell 204, a condenser lens 213 for condensing fluorescence light and a CCD area image sensor were disposed.

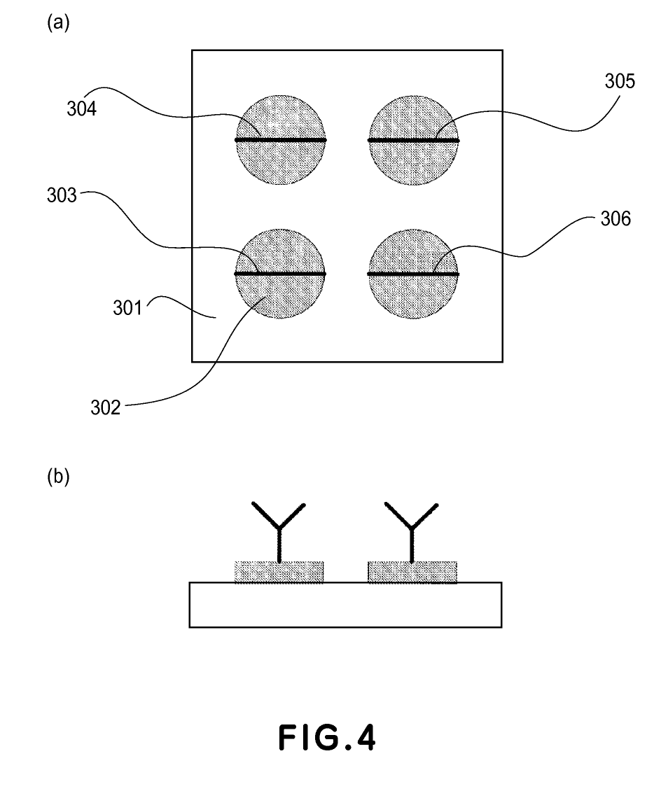

Detailed description will be made with reference to FIGS. 4(a) and 4(b).

FIGS. 4(a) and 4(b) are a conceptual plan view and a conceptual sectional view, respectively, showing such a state that a metal array 302 comprising metal films (dots) is formed on a substrate 301 and ligands including an anti-CEA antibody 303, an anti-AFP antibody 305, an anti-PSA antibody 304, and an anti-PAP antibody 306 are fixed. As a method of fixing these antibodies (ligands) on the metal array 302, a method in which avidin is adsorbed on the metal substrate through SH group and an antibody is modified with biotin to be fixed by avidin-biotin was employed.

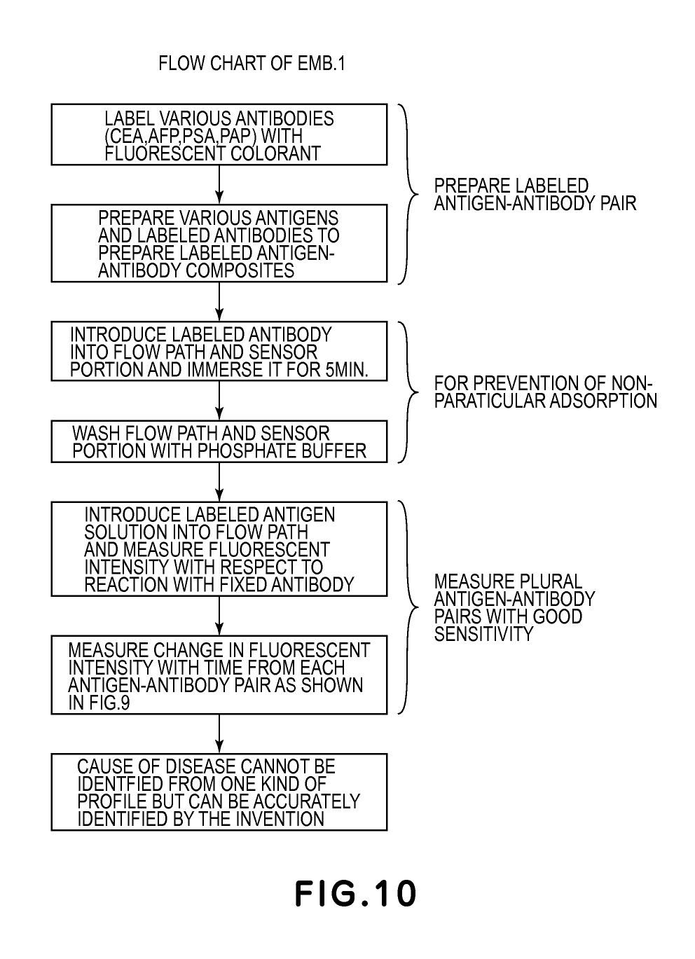

The substrate 301 was set in a fluorescence analysis apparatus and subjected to measurement in accordance with a protocol (procedure) shown in FIG. 10.

(1) The above described 4 species of antibodies which had been fluorescence-labeled with Cy5 dye were caused to react with the respective antigens to prepare labeled antigens.

(2) The fluorescence-labeled antibodies were introduced into a flow path and incubated for 5 minutes.

(3) The labeled antibodies were taken out of the flow path and washed with phosphate buffer.

(4) A specimen in which labeled antigens of CEOA, PSA, AFP and PAP were added in mixture was introduced into the flow path.

(5) Through laser light irradiation, a surface plasmon resonance curve and a change with time in fluorescent signal were detected from respective metal film dots.

When data of the change with time in fluorescence signal were plotted, a result as shown in FIG. 9 was obtained. Further, an amount of reaction and fixation of ligand-target substance composite can be estimated from fluorescence intensity after completion of adsorption reaction and dissociation and the respective reaction (fixation) amount are shown in Table 1.

TABLE 1

Species

| | CEA | PSA | AFP | PAP |
|---|---|---|---|---|
| Amount (ng/ml) | 4.5 | 3.5 | 7.4 | 2.5 |

By comparing the profile shown in FIG. 9 and the reaction amounts shown in Table 1 with those stored in data base, it is possible to specify is site of cancer with high accuracy.

According to this embodiment, it is possible to measure a plurality of antigen-antibody reactions at the same time. Further, it is possible to measure a change in fluorescence intensity with time by using an electric field generated by plasmon resonance. As a result, it is possible to concurrently measure the plurality of antigen-antibody reactions to obtain values from the reaction profile with time and the reaction amounts.

### Embodiment 2

Hybridization measurement of DNA was performed by using the optical system employed in Embodiment 1.

On the surface of each metal arrays, streptavidin was deposited, and 4 species of biotin-modified 20-mer DNA were adsorbed to prepare a nucleic acid sensor.

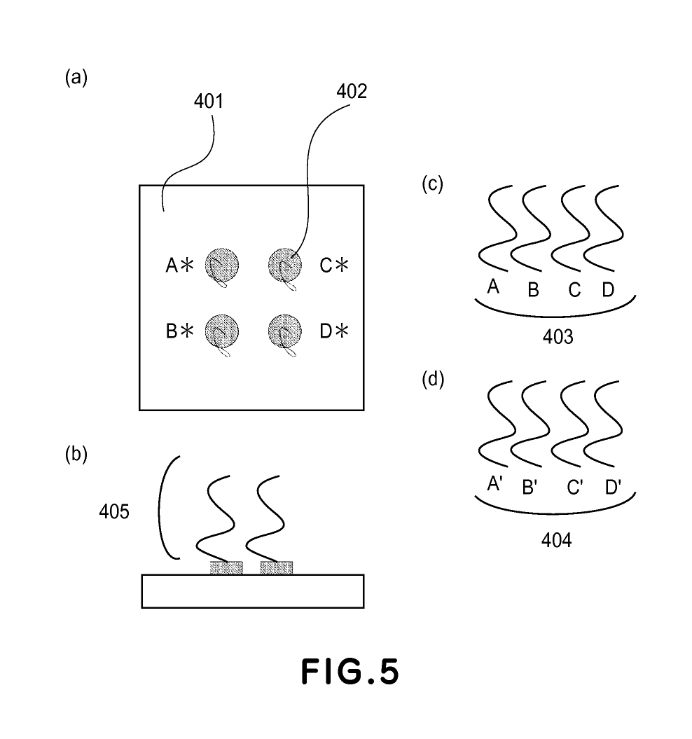

Detailed description will be made with reference to FIGS. 5(a) to 5(d).

As shown in FIG. 5(a) (plan view), on a 2×2 metal array 402 formed on a substrate 401, 4 species of biotin-modified 20-mer DNAs were fixed and subjected to the following protocol (procedure).

Fixation of different ligands on the metal array was performed according to a method described in U.S. Pat. No. 6,194,223.

(1) A specimen liquid in which four species (A*, B*, C* and D*) of composites 403, fluorescence-labeled with Cy5 dye, comprising DNA 405 (probe P) having fixed base sequence as shown in FIG. 5(b) and corresponding ligand DNAs (target T1, 20-mer as shown in FIG. 4(c) and four species of 20-mer DNAs (target T2; A′, B′, C′ and D′), fluorescence-labeled with Cy3 dye, different in base sequence only by one as shown in FIG. 5(d) are added in mixture.

(2) The specimen liquid is introduced into a flow path and incubated for 5 minutes.

(3) The specimen is taken out of the flow path and washed with phosphate buffer.

(4) The phosphate buffer is injected into the flow path.

After the operation of (4), when plasmon resonance and fluorescence intensity were measured through irradiation with laser light, the plasmon resonance curve was not changed before and after the hybridization reaction of DNA. On the other hand, in the measurement of fluorescence, it was confirmed that sensitive measurement on the order of 1 nM as a concentration of DNA was performed. When fluorescence spectrum was measured by a spectrometer (not shown), it was confirmed that only a dye having a peak wavelength of approximately 670 nm produced fluorescence. As a result, it was confirmed that only the DNA of T1 was particularly connected.

As described hereinabove, according to the present invention, it is possible to concurrently detect fluorescence immunoassay and surface plasmon resonance with respect to a plurality of ligand-target substance composites. Further, on the basis of detection results, it is possible to effect imaging. IN addition, it is possible to provide a detection apparatus capable of performing the above described detection, so that it becomes possible to perform screening for measuring a plurality of items as the same time.

While the invention has been described with reference to the structures disclosed herein, it is not confined to the details set forth and this application is intended to cover such modifications or changes as may come within the purpose of the improvements or the scope of the following claims.

This application claims priority from Japanese Patent Application No. 023517/2005 filed Jan. 31, 2005, which is hereby incorporated by reference.

What is claimed is:

1. A detection apparatus, comprising:
a substrate;
a metal array, formed on said substrate, comprising a plurality of metal films;
a first ligand, disposed on the metal films, for catching a first chemical substance;
a second ligand, disposed on the metal films, for catching a second chemical substance different from the first chemical substance;
a first optical system disposed on a first surface of said substrate; and
a second optical system disposed on a second surface, of said substrate, different from the first surface;
wherein said first optical system includes first detection means comprising a two-dimensional optical sensor for detecting reflected light when a ligand-target substance composite comprising the metal films, said first and second ligands, and the first and second chemical substances is irradiated with emitted light from a first light source; and
wherein said second optical system includes second detection means comprising a two-dimensional optical sensor for detecting fluorescence from the ligand-target substance composite excited when the ligand-target substance composite is irradiated with the emitted light from the first light source.

2. An apparatus according to claim 1, wherein each of said first and second ligands is protein.

3. An apparatus according to claim 1, wherein each of said first and second ligands is nucleic acid.

4. An apparatus according to claim 1, wherein said detection apparatus effects scanning so that an angle between an optical axis of the first light source and said substrate is kept equal to an angle between an optical axis of said first optical system and said substrate.

5. A detection method, comprising:
a step of catching a first chemical substance by a first ligand fixed on a plurality of metal films formed on a substrate,
a step of catching a second chemical substance, different from the first chemical substance, by a second ligand fixed on the plurality of metal films,
a step of causing emitted light from a first light source to enter a ligand-target substance composite comprising the substrate, the metal films, and the first and second chemical substances,
a first detection step of detecting reflected light from the ligand-target substance composite by a two-dimensional optical sensor, and
a second detection step of detecting fluorescence from the ligand-target substance composite excited by the emitted light caused to enter the ligand-target substance composite.

6. A method according to claim 5, wherein said detection apparatus effects scanning so that an angle between an optical axis of the first light source and said substrate is kept equal to an angle between an optical axis of said first optical system and said substrate.
